(19) United States
(12) Patent Application Publication
(10) Pub. No.: US 2010/0172795 A1
(43) Pub. Date: Jul. 8, 2010

(54) METHOD AND APPARATUS FOR THE TREATMENT OF METAL CONTAINERS, SUCH AS BEVERAGE CANS, IN A BEVERAGE CAN FILLING PLANT

(76) Inventor: Wilhelm LOTHAR, Karben (DE)

Correspondence Address: NILS H. LJUNGMAN & ASSOCIATES, P. O. BOX 130, GREENSBURG, PA, 15601-0130, US

(21) Appl. No.: 12/501,564

(22) Filed: Jul. 13, 2009

Related U.S. Application Data
(63) Continuation-in-part of application No. PCT/EP2007/011235, filed on Dec. 20, 2007.

(30) Foreign Application Priority Data
Jan. 13, 2007 (DE) 102007001970.1

(51) Int. Cl. A61L 2/18; A61L 2/16

(52) U.S. Cl. 422/28; 422/302

(57) ABSTRACT

A method and apparatus for the treatment of metal containers, such as beverage cans, in a beverage can filling plant. The abstract of the disclosure is submitted herewith as required by 37 C.F.R. §1.72(b). As stated in 37 C.F.R. §1.72(b): A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims. Therefore, any statements made relating to the abstract are not intended to limit the claims in any manner and should not be interpreted as limiting the claims in any manner.

### CONTINUING APPLICATION DATA

This application is a Continuation-In-Part application of International Patent Application No. PCT/EP2007/011235, filed on Dec. 20, 2007, which claims priority from Federal Republic of Germany Patent Application No. 10 2007 001 970.1, filed on Jan. 13, 2007. International Patent Application No. PCT/EP2007/011235 was pending as of the filing date of this application. The United States was an elected state in International Patent Application No. PCT/EP2007/011235.

### BACKGROUND

1. Technical Field

The present application relates to a method and apparatus for the treatment of metal containers, such as beverage cans, in a beverage can filling plant.

2. Background Information

Background information is for informational purposes only and does not necessarily admit that subsequently mentioned information and publications are prior art.

A beverage bottling plant for filling bottles with a liquid beverage filling material can possibly comprise a beverage filling machine, which is often a rotary filling machine, with a plurality of beverage filling positions, each beverage filling position having a beverage filling device for filling bottles with liquid beverage filling material. The filling devices may have an apparatus designed to introduce a predetermined volume of liquid beverage filling material into the interior of bottles to a substantially predetermined level of liquid beverage filling material.

Some beverage bottling plants may possibly comprise filling arrangements that receive a liquid beverage material from a toroidal or annular vessel, in which a supply of liquid beverage material is stored under pressure by a gas. The toroidal vessel may also be connected to at least one external reservoir or supply of liquid beverage material by a conduit or supply line. In some circumstances it may even be possible that a beverage bottling plant has two external supply reservoirs, each of which may be configured to store either the same liquid beverage product or different products. These reservoirs could possibly be connected to the toroidal or annular vessel by corresponding supply lines, conduits, or other arrangements. It is also possible that the external supply reservoirs could be in the form of simple storage tanks, or in the form of liquid beverage product mixers.

A wide variety of types of filling elements are used in filling machines in beverage bottling or container filling plants for dispensing a liquid product into bottles, cans or similar containers, including but not limited to filling processes that are carried out under counterpressure for the bottling of carbonated beverages. The apparatus designed to introduce a predetermined flow of liquid beverage filling material further comprises an apparatus that is designed to terminate the filling of the beverage bottles upon the liquid beverage filling material reaching the predetermined level in bottles. There may also be provided a conveyer arrangement that is designed to move bottles, for example, from an inspecting machine to the filling machine.

After a filling process has been completed, the filled beverage bottles are transported or conveyed to a closing machine, which is often a rotary closing machine. A revolving or rotary machine comprises a rotor, which revolves around a central, vertical machine axis. There may further be provided a conveyer arrangement configured to transfer filled bottles from the filling machine to the closing station. A transporting or conveying arrangement can utilize transport star wheels as well as linear conveyors. A closing machine closes bottles by applying a closure, such as a screw-top cap or a bottle cork, to a corresponding bottle mouth. Closed bottles are then usually conveyed to an information adding arrangement, wherein information, such as a product name or a manufacturer's information or logo, is applied to a bottle. A closing station and information adding arrangement may be connected by a corresponding conveyer arrangement. Bottles are then sorted and packaged for shipment out of the plant.

Many beverage bottling plants may also possibly comprise a rinsing arrangement or rinsing station to which new, non-return and/or even return bottles are fed, prior to being filled, by a conveyer arrangement, which can be a linear conveyor or a combination of a linear conveyor and a starwheel. Downstream of the rinsing arrangement or rinsing station, in the direction of travel, rinsed bottles are then transported to the beverage filling machine by a second conveyer arrangement that is formed, for example, by one or more starwheels that introduce bottles into the beverage filling machine.

It is a further possibility that a beverage bottling plant for filling bottles with a liquid beverage filling material can be controlled by a central control arrangement, which could be, for example, a computerized control system that monitors and controls the operation of the various stations and mechanisms of the beverage bottling plant.

The sterilization of packaging containers of the type used in the food and beverage industry, for example, is becoming increasingly important because a long shelf life of the contents of the container without the addition of preservatives or a subsequent treatment, e.g. by pasteurization, is becoming increasingly unattractive on account of its negative effect on the packaged product.

To prevent, restrict, and/or minimize this, for example, aseptic filling machines are used to fill bottles. For this purpose, the entire bottling plant or a portion of the bottling plant is operated in a sterile environment. To achieve this sterile environment, the plant is enclosed in a housing, the interior is sterilized and kept as sterile as possible, and steps are taken to essentially ensure or promote that the air and all or most other substances that are introduced into the interior are practically sterile. An essential component of this process is also that the interior of the containers to be filled is also sterilized. On account of various problems that occur with the use of metal containers, it has not yet been possible to satisfactorily realize plants for use with metal containers to be filled, such as beverage cans, for example.

A number of different methods are available for the sterilization of such containers. Metal containers are currently sterilized, if at all, by steam sterilization or in a batch process. Otherwise, they are not used for the aseptic filling process described above.

Additional sterilization methods use different chemical sterilization media. One method is sterilization with hydrogen peroxide.

### OBJECT OR OBJECTS

An object of the present application is to make available a method for the sterilization of metal containers, in one possible embodiment cans, which makes it possible to achieve good sterilization and can simultaneously or substantially simultaneously be used in plants with a high production rate.

### SUMMARY

The present application teaches that this object is accomplished by a method for the sterilization of metal containers, in one possible embodiment cans, wherein a sterilization medium in liquid and/or vapor form is introduced into the interior of the containers, and that then the sterilization medium is activated and the sterilization process is started. The present application also teaches that this object may be accomplished by an apparatus for the sterilization of metal containers, wherein a device is provided for the introduction of a sterilization medium in the form of a liquid and/or vapor into the interior of the containers, as is a device for the activation of the sterilization medium. The present application also teaches that this object may be accomplished by an apparatus for the sterilization of metal containers, wherein for the activation of a sterilization process, at least one induction coil device is provided for the inductive heating of the containers.

Developments of the present application are described according to the present application.

With the method according to the present application, a sterilization medium in liquid and/or vapor form is first introduced into the interior of each container. In one possible embodiment of the present application, hydrogen peroxide (H₂O₂) as a disinfectant is used. The disinfectant is first atomized, for example, mixed with an air stream which in one possible embodiment comprises sterile air, and the mixture is then completely evaporated in a vaporizer. That results in an air current which is enriched, for example, to saturation with H₂O₂ in vapor form, which is subsequently introduced into the interior of the container. There it condenses on the container wall, which is relatively colder than the mixture, where if forms a continuous liquid film.

The introduction of the sterilization agent is achieved, for example, with a carousel-type arrangement, in which the cans circulate in a rotating arrangement and are thereby filled with the sterilization agent. Then they are removed from the turntable and transported to the activation devices.

The method according to the present application teaches that the sterilization medium is activated and thus the sterilization method is started, which in the case of the use of H₂O₂ is achieved by the addition of a specified quantity of heat. As the condensed H₂O₂ is heated beyond a specified threshold, a decomposition process begins which decomposes the H₂O₂. In the course of the decomposition process, free radicals are produced in the form of atomic O and HO groups, among others, which react with any impurities that may be present and thereby perform the actual sterilization, whereby water and some decomposition residues are left over as the decomposition products.

In some configurations of the present application, the quantity of heat required and/or desired for activation can be provided in a number of different ways. First, heated air, and sterile heated air in one possible embodiment, can be introduced in the vicinity of the liquid film. As a result the condensed H₂O₂ is heated beyond the activation point, so that the decomposition process and thus the actual sterilization can begin.

In an additional realization of the present application, the container itself is heated so that the condensed H₂O₂ is also heated beyond the activation point and the sterilization process begins. In this realization, the container is heated inductively. In other words, the container is heated by a suitable arrangement past which the container is guided, for example an induction coil or similar device, by the generation of eddy or ring currents in the metal container itself.

A reaction line can be used in one possible embodiment for this purpose, in which after the introduction of the sterilization agent, the containers are moved past the inductive heating devices and heated. Then the container travels through a tunnel-shaped device which is flushed by a current of air in the direction opposite to the direction of transport, in one possible embodiment with the use of sterile air, so that the gaseous decomposition products that are formed during the decomposition of the sterilization medium can be absorbed and transported away. At the end of this section, the containers are sterilized and can be filled with the product.

In an alternative realization, a rotating arrangement is also selected for the activation, so that, similar to the introduction of the heated sterilization agent, in one possible embodiment sterile air, into the interior of the containers, where the sterilization process is initiated. The dimensions of the carousel-like arrangements must or may be be selected so that the sterilization process has been essentially completed by the completion of one revolution. By means of corresponding ventilation measures, it is also essentially ensured or promoted that the decomposition products are removed. After the completion of the sterilization process, the sterilized containers are then delivered to the filling devices. Depending on the size and output of the plants, it may also be appropriate to use a plurality of carousels connected in series one after another so that the process can be completed.

In one realization the present application teaches that the covers provided for the closing of the containers are also sterilized. This sterilization takes place analogous to the method described above by applying a sterilization agent in liquid or vapor form to the covers and then initiating the sterilization process by supplying heat. In this realization, a linear arrangement is provided in which the covers are first separated from one another and then, on a conveyor line, are initially wetted or sprayed with the sterilization agent and then the decomposition process is initiated by the addition of heat. The layout and dimensions of the line are thereby selected so that the decomposition process is completed and the covers are then delivered directly to the closer, after which the filled containers are closed with the covers.

In one possible embodiment according to the present application, it may be possible to rinse the containers and/or the covers with sterile water once more before the filling or closing.

In at least one possible embodiment according to the present application, the sterilizing machine, filling machine, and/or closing machine can process beverage cans at a rate of approximately fifty thousand cans per hour to seventy thousand cans per hour. In other embodiments, the machines may be configured to handle cans at either a higher rate or a lower rate.

The above-discussed embodiments of the present invention will be described further herein below. When the word “invention” or “embodiment of the invention” is used in this specification, the word “invention” or “embodiment of the invention” includes “inventions” or “embodiments of the invention”, that is the plural of “invention” or “embodiment of the invention”. By stating “invention” or “embodiment of the invention”, the Applicant does not in any way admit that the present application does not include more than one patentably and non-obviously distinct invention, and maintains that this application may include more than one patentably and non-obviously distinct invention. The Applicant hereby asserts that the disclosure of this application may include more than one invention, and, in the event that there is more than one invention, that these inventions may be patentable and non-obvious one with respect to the other.

### BRIEF DESCRIPTION OF THE DRAWINGS

The present application is explained in greater detail below with reference to the accompanying drawings, in which:

FIG. 1 is a schematic overhead view of a first realization of the present application;

FIG. 2 is a schematic overhead view of an additional realization of the present application;

FIG. 3 shows a block diagram of one possible embodiment of the present application;

FIG. 4 shows a block diagram of another possible embodiment of the present application; and

FIG. 5 shows schematically the main components of one possible embodiment example of a system for filling containers, specifically, a beverage bottling plant for filling bottles with at least one liquid beverage, in accordance with at least one possible embodiment, in which system or plant could possibly be utilized at least one aspect, or several aspects, of the embodiments disclosed herein.

### DESCRIPTION OF EMBODIMENT OR EMBODIMENTS

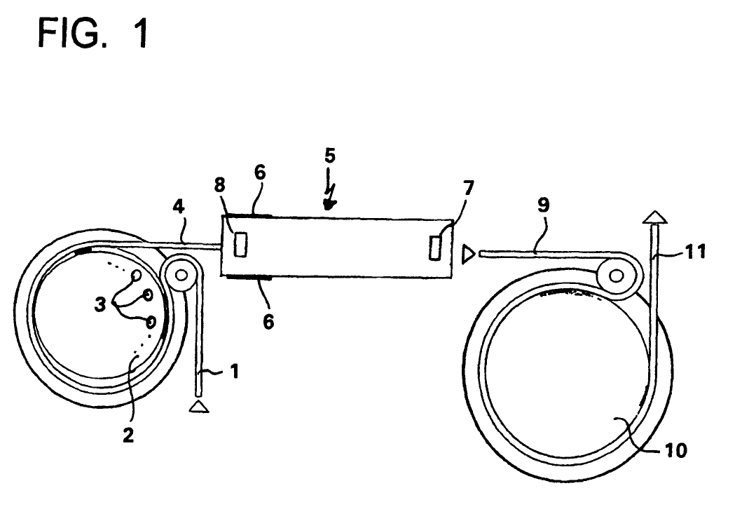

FIG. 1 shows an apparatus for the sterilization of metal containers, in one possible embodiment cans for beverages, in a first realization. The purpose of the apparatus is in one possible embodiment to sterilize beverage cans so that the cans can be filled using an aseptic filling process and so that the contents have a long shelf life without the need or desire to use preservatives or pasteurization processes or similar treatments.

For this purpose, the empty metal containers are transferred from a conveyor device 1 to a sterilization carousel 2. There the containers continue to be rotated in a counterclockwise direction. A sterilizer carousel that has a plurality of sterilization heads 1 rotates together with the containers.

By means of the sterilization heads 3, a mixture of H₂O₂ vapor and sterile air is introduced into the interior of the containers. For this purpose, first liquid H₂O₂ is finely atomized and mixed with a flow of sterile air. This mixture is then transported through vaporizer heads which vaporize the liquid components of the H₂O₂ and thereby produce a saturated mixture of H₂O₂ vapor and sterile air. This mixture has a higher temperature than the metal containers into which it is introduced, so that the H₂O₂ condenses out of the mixture on the cold interior walls of the container, where it forms a uniform film of liquid. The sizing of the sterilizer carousel is selected so that a sufficient film of liquid is present in the interior of the container when one revolution has been completed.

In at least one possible embodiment of the present application, the sterilization heads 3 inject into the beverage cans a sufficient amount of hydrogen peroxide to condense on substantially all the inner surfaces of the cans. A sufficient amount of time elapses to allow the atomized hydrogen peroxide to condense on substantially all the inner surfaces of the beverage can before the beverage can is moved from the sterilization carousel or sterilization machine 2 and into the reaction line 5. The reaction line or reaction tunnel 5 is sufficiently long to permit the heat from the induction coil arrangements 6 to heat the cans and activate the hydrogen peroxide and therefore sufficiently sterilize and/or treat the cans before the cans are filled with a filler 10. A sufficient period of time passes for the activated hydrogen peroxide to destroy any contaminants in the cans, such as bacteria.

Then the containers are fed by an additional transport device 4 to a reaction line 5. This reaction line is realized in the form of a tunnel. Initially, two induction coil arrangements 6 are installed which heat the metal containers traveling past them by the induction of eddy currents. This method is used when the cans are at least partly magnetizable.

As a result of the heating of the cans, the sterilization medium which is inside the cans in the form of a uniform film is heated beyond its activation point, whereupon, when H₂O₂ is used, a decomposition process begins in which free radicals such as 0 and HO groups are formed, among other things, via a plurality of intermediate stages. These free radicals react with any impurities that may be present and thereby perform the actual sterilization process. Upon the completion of the process, the remaining products are primarily water with a few residues of the decomposition process which are biologically inactive, however, so that the interior surfaces of the container treated with them are essentially sterile.

After the initial activation by the heating of the cans with the induction coil devices 6, the decomposition process proceeds on its own. The length of the reaction line 5 must or should thereby be sized so that the process is essentially completed by the end of the line. At the end of the tunnel, there is an air feed 7 which introduces sterile air into the interior of the tunnel. At the beginning of the tunnel, there is an exhaust device 8 which sucks the air out of the interior of the tunnel. The result is an airflow which is directed opposite to the direction of travel of the containers and which absorbs any gaseous decomposition products that may be formed and removes them from the interior of the tunnel.

At the end of the tunnel, the sterilized containers are fed by an additional transport device 9 to the filler 10, by which the sterilized containers are filled in the known manner with the product being canned. Then the filled containers are fed by an additional transport device 11 to a closer which is also of the type known from the prior art.

The present application teaches that the covers used for the closing process are also sterilized, at least on the inside.

For this purpose, a cover sterilization device which is not illustrated in any further detail is provided, which in a linear section, as describes above, first applies a sufficiently uniform liquid film of H₂O₂ to the cover and then activates the film by the addition of heat in the form of heated sterile air. After the completion of the process, the covers that have been sterilized in this manner are then immediately or substantially immediately delivered to the closer, where they are placed on the filled metal containers, as a result of which the containers are closed.

Alternatively, both the containers and the covers can be rinsed again with sterile water.

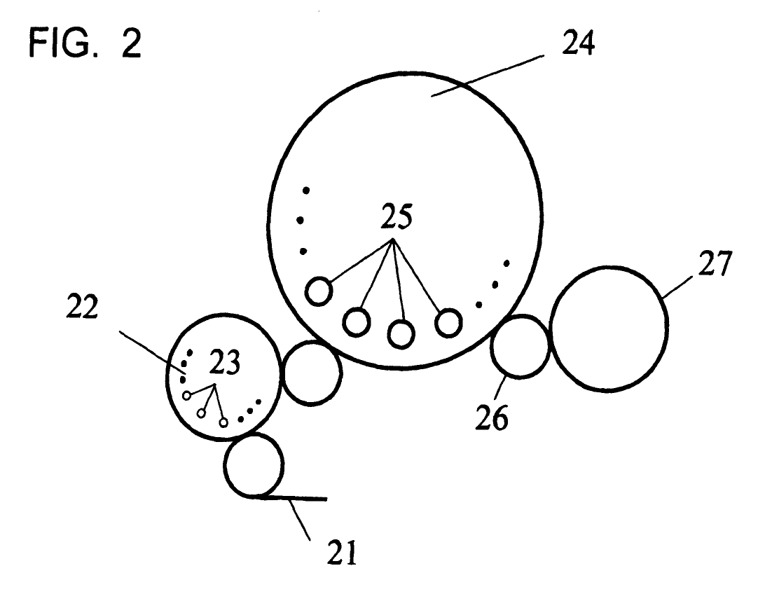

An alternative realization of the sterilization plant according to the present application is schematically illustrated in FIG. 2.

Here, too, the containers are transferred a conveyor device 21 onto a sterilizer carousel 22 and are provided in the manner described above by means of vaporizer heads 23 and the feed of a mixture of H₂O₂ vapor and sterile air with a uniform condensation film of the sterilization medium on the inside. Then the containers are transferred to a reaction carousel 24, where they also revolve on a circular path. On the reaction carousel 24 there are feed nozzles 25 which blow heated sterile air into the interior of the containers and thus heat the H₂O₂ beyond the activation point so that the decomposition process described above begins, at the end of which the containers are sterilized. The size of the reaction carousel 24 must or should thereby be selected so that the required or desired cycle time of the containers from their placement on the carousel to their removal from the carousel equals the time required or desired for the completion of the sterilization process. Depending on the realization, the heated air can be introduced once at the beginning of the process or an airflow can be maintained during the decomposition process to accelerate the process and to remove the decomposition products from the interior of the container.

After the completion of the sterilization process, the containers are fed from the reaction carousel 24 via a transport star wheel 26 to the filler 27. Here, the product is introduced into the containers in the known manner.

Then, analogous to the process described above, the cans are delivered to a closer where they are closed with a cover which is sterilized in the manner described above.

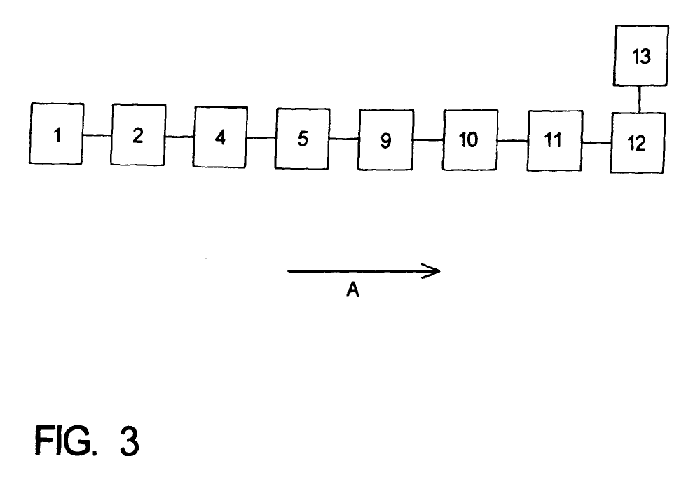

FIG. 3 shows a block diagram of one possible embodiment of the present application. First, the cans may be transported, in a direction of transport A, by a conveyor device 1 to a sterilization carousel or sterilization machine or treating machine 2. Once the cans are treated, they may be transported by an additional transport device 4 to a reaction line 5. The hydrogen peroxide may be activated and the can may be sterilized in the reaction line 5. An additional transport device 9 may feed the cans into a filler 10, which fills the sterilized cans. An additional transport device 11 may then transport the cans to a closing machine or closing carousel 12. A cover sterilization machine 13 is configured to sterilize at least the inside of the covers. The covers are then fed into the closing machine 12, which is configured to place the sterilized covers on the sterilized cans and/or seal the cans.

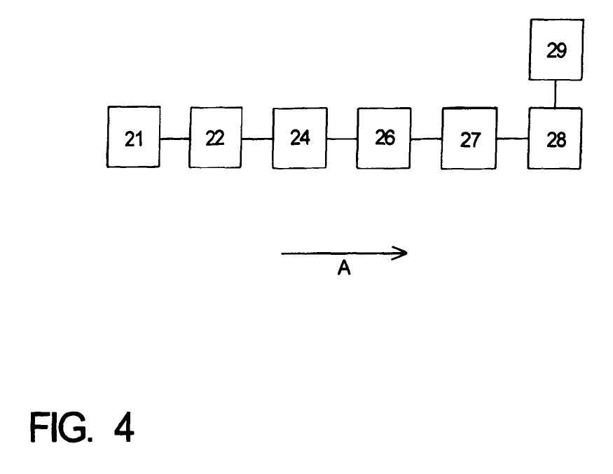

FIG. 4 shows a block diagram of an additional embodiment according to the present application. As seen in FIG. 4, cans may be transported, in a direction of transport A, by a conveyor device 21 to a sterilizer carousel or sterilization machine 22. The cans may be sterilized and then transported to a reaction carousel or reaction machine 24, in which the hydrogen peroxide may be activated and the cans may be sterilized. A transport star wheel 26 feeds the cans from the reaction machine 24 to a filler 27. The sterilized cans may be filled by the filler 27, and then may be transported to a can closing machine 28. A cover sterilization machine 29 is configured to sterilize at least the inside of the covers. The covers are then fed into the closing machine 28, which is configured to place the sterilized covers on the sterilized cans and/or seal the cans.

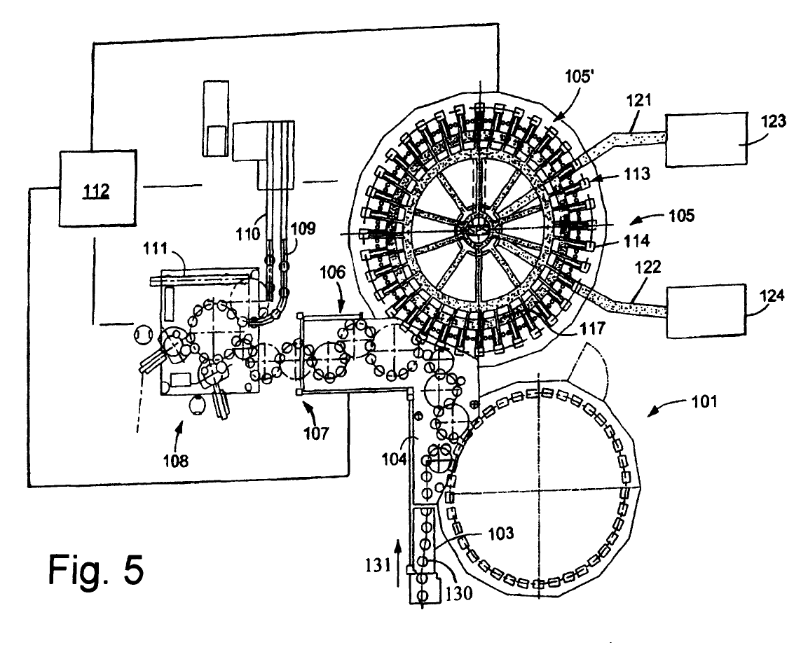

FIG. 5 shows a rinsing arrangement or rinsing station 101, to which the containers, namely bottles 130, are fed in the direction of travel as indicated by the arrow 131, by a first conveyer arrangement 103, which can be a linear conveyor or a combination of a linear conveyor and a starwheel. Downstream of the rinsing arrangement or rinsing station 101, in the direction of travel as indicated by the arrow 131, the rinsed bottles 130 are transported to a beverage filling machine 105 by a second conveyer arrangement 104 that is formed, for example, by one or more starwheels that introduce bottles 130 into the beverage filling machine 105.

The beverage filling machine 105 shown is of a revolving or rotary design, with a rotor 105′, which revolves around a central, vertical machine axis. The rotor 105′ is designed to receive and hold the bottles 130 for filling at a plurality of filling positions 113 located about the periphery of the rotor 105′. At each of the filling positions 103 is located a filling arrangement 114 having at least one filling device, element, apparatus, or valve. The filling arrangements 114 are designed to introduce a predetermined volume or amount of liquid beverage into the interior of the bottles 130 to a predetermined or desired level.

The filling arrangements 114 receive the liquid beverage material from a toroidal or annular vessel 117, in which a supply of liquid beverage material is stored under pressure by a gas. The toroidal vessel 117 is a component, for example, of the revolving rotor 105′. The toroidal vessel 117 can be connected by means of a rotary coupling or a coupling that permits rotation. The toroidal vessel 117 is also connected to at least one external reservoir or supply of liquid beverage material by a conduit or supply line. In the embodiment shown in FIG. 5, there are two external supply reservoirs 123 and 124, each of which is configured to store either the same liquid beverage product or different products. These reservoirs 123, 124 are connected to the toroidal or annular vessel 117 by corresponding supply lines, conduits, or arrangements 121 and 122. The external supply reservoirs 123, 124 could be in the form of simple storage tanks, or in the form of liquid beverage product mixers, in at least one possible embodiment.

As well as the more typical filling machines having one toroidal vessel, it is possible that in at least one possible embodiment there could be a second toroidal or annular vessel which contains a second product. In this case, each filling arrangement 114 could be connected by separate connections to each of the two toroidal vessels and have two individually-controllable fluid or control valves, so that in each bottle 130, the first product or the second product can be filled by means of an appropriate control of the filling product or fluid valves.

Downstream of the beverage filling machine 105, in the direction of travel of the bottles 130, there can be a beverage bottle closing arrangement or closing station 106 which closes or caps the bottles 130. The beverage bottle closing arrangement or closing station 106 can be connected by a third conveyer arrangement 107 to a beverage bottle labeling arrangement or labeling station 108. The third conveyor arrangement may be formed, for example, by a plurality of starwheels, or may also include a linear conveyor device.

In the illustrated embodiment, the beverage bottle labeling arrangement or labeling station 108 has at least one labeling unit, device, or module, for applying labels to bottles 130. In the embodiment shown, the labeling arrangement 108 is connected by a starwheel conveyer structure to three output conveyer arrangements: a first output conveyer arrangement 109, a second output conveyer arrangement 110, and a third output conveyer arrangement 111, all of which convey filled, closed, and labeled bottles 130 to different locations.

The first output conveyer arrangement 109, in the embodiment shown, is designed to convey bottles 130 that are filled with a first type of liquid beverage supplied by, for example, the supply reservoir 123. The second output conveyer arrangement 110, in the embodiment shown, is designed to convey bottles 130 that are filled with a second type of liquid beverage supplied by, for example, the supply reservoir 124. The third output conveyer arrangement 111, in the embodiment shown, is designed to convey incorrectly labeled bottles 130. To further explain, the labeling arrangement 108 can comprise at least one beverage bottle inspection or monitoring device that inspects or monitors the location of labels on the bottles 130 to determine if the labels have been correctly placed or aligned on the bottles 130. The third output conveyer arrangement 111 removes any bottles 130 which have been incorrectly labeled as determined by the inspecting device.

The beverage bottling plant can be controlled by a central control arrangement 112, which could be, for example, computerized control system that monitors and controls the operation of the various stations and mechanisms of the beverage bottling plant.

The present application is not limited to the possible embodiment described above, but can be the subject of numerous modifications and variations without thereby going beyond the teaching of the present application.

The metal containers do not have to be beverage cans, but can also be suitable for other applications in the food and beverage industry. Containers for use in medicine or pharmacy, for example, can also be sterilized in the manner described above. The sterilization medium can be introduced into the containers in a number of different ways. In addition to vaporization by means of vaporizer heads, the use of spray heads or even the introduction of liquid media into the interior of the container are conceivable. In addition to the activation by the inductive heating of the containers described above or by the introduction of hot air, other methods to accomplish the same purpose are also conceivable, such as, for example, infrared radiation. The realization of the overall plant is also open to a wide range of variations, such as the configuration of the individual parts of the plant or their layout, for example.

One feature or aspect of an embodiment is believed at the time of the filing of this patent application to possibly reside broadly in a method for the sterilization of metal containers, in one possible embodiment cans, wherein a sterilization medium in liquid and/or vapor form is introduced into the interior of the containers, and that in connection therewith the sterilization medium is activated and the sterilization process is started.

Another feature or aspect of an embodiment is believed at the time of the filing of this patent application to possibly reside broadly in the method, wherein the covers provided for the closing of the containers are sterilized.

Yet another feature or aspect of an embodiment is believed at the time of the filing of this patent application to possibly reside broadly in the method, wherein a sterilization medium in liquid and/or vapor form is applied to the cover, and that then the sterilization medium is activated, thereby starting the sterilization process.

Still another feature or aspect of an embodiment is believed at the time of the filing of this patent application to possibly reside broadly in the method, wherein the introduction into the containers and/or the application to the covers is performed with sterilization medium in liquid and/or vapor form, in one possible embodiment sterile air.

A further feature or aspect of an embodiment is believed at the time of the filing of this patent application to possibly reside broadly in the method, wherein hydrogen peroxide (H₂O₂) in liquid and/or vapor form is used as the sterilization medium in liquid and/or vapor form.

Another feature or aspect of an embodiment is believed at the time of the filing of this patent application to possibly reside broadly in the method, wherein the sterilization medium is heated for its activation.

Yet another feature or aspect of an embodiment is believed at the time of the filing of this patent application to possibly reside broadly in the method, wherein the sterilization medium is activated by the supply of heated air, in one possible embodiment of heated sterile air.

Still another feature or aspect of an embodiment is believed at the time of the filing of this patent application to possibly reside broadly in the method, wherein the sterilization medium is activated by a heating of the containers and/or of the covers.

A further feature or aspect of an embodiment is believed at the time of the filing of this patent application to possibly reside broadly in the method, wherein the containers and/or covers are heated inductively.

Another feature or aspect of an embodiment is believed at the time of the filing of this patent application to possibly reside broadly in the method, wherein air, in one possible embodiment sterile air, is used for the removal of the decomposition products that are formed during the sterilization products and or sterilization medium residue.

Yet another feature or aspect of an embodiment is believed at the time of the filing of this patent application to possibly reside broadly in an apparatus for the sterilization of metal containers, wherein a device is provided for the introduction of a sterilization medium in the form of a liquid and/or vapor into the interior of the containers, and a device for the activation of the sterilization medium is provided.

Still another feature or aspect of an embodiment is believed at the time of the filing of this patent application to possibly reside broadly in the apparatus, wherein an additional sterilization device is provided for the covers provided for the closing of the containers.

A further feature or aspect of an embodiment is believed at the time of the filing of this patent application to possibly reside broadly in the apparatus, wherein a distributor device for a mixture of hydrogen peroxide (H₂O₂) in liquid and/or vapor form and air is provided as the device for the introduction of a sterilization medium in liquid and/or vapor form into the interior of the containers.

One feature or aspect of an embodiment is believed at the time of the filing of this patent application to possibly reside broadly in the apparatus, wherein for the activation of the sterilization medium, an apparatus is provided for the heating of the containers.

Another feature or aspect of an embodiment is believed at the time of the filing of this patent application to possibly reside broadly in the apparatus, wherein for the activation of the sterilization medium, an apparatus is provided for the introduction of heated air, in one possible embodiment of sterile air, into the interior of the container.

Yet another feature or aspect of an embodiment is believed at the time of the filing of this patent application to possibly reside broadly in the apparatus, wherein at least one induction coil device 6 for the inductive heating of the containers is provided for the activation of the sterilization medium.

Still another feature or aspect of an embodiment is believed at the time of the filing of this patent application to possibly reside broadly in the apparatus, wherein a conveyor device is provided for the transport of the containers to be sterilized to the at least one induction coil device 6.

A further feature or aspect of an embodiment is believed at the time of the filing of this patent application to possibly reside broadly in the apparatus, wherein following the at least one induction coil device 6, a reaction line 5 is provided for the performance of the sterilization process and an air current, in one possible embodiment of sterile air, is provided flowing in the direction opposite to the direction of transport.

Another feature or aspect of an embodiment is believed at the time of the filing of this patent application to possibly reside broadly in an apparatus for the sterilization of metal containers, wherein for the activation of a sterilization process, at least one induction coil device 6 is provided for the inductive heating of the containers.

Yet another feature or aspect of an embodiment is believed at the time of the filing of this patent application to possibly reside broadly in the apparatus, wherein a conveyor device is provided for the transport of the containers to be sterilized past the at least one induction coil device 6.

Still another feature or aspect of an embodiment is believed at the time of the filing of this patent application to possibly reside broadly in the apparatus, wherein following the at least one induction coil device 6, a reaction line 5 is provided for the performance of the sterilization process and an air current, in one possible embodiment of sterile air, is provided flowing opposite to the direction of transport.

A further feature or aspect of an embodiment is believed at the time of the filing of this patent application to possibly reside broadly in the apparatus, wherein an apparatus is provided for the introduction of a sterilization medium that can be activated by heat into the interior of the containers.

The components disclosed in the various publications, disclosed or incorporated by reference herein, may possibly be used in possible embodiments of the present invention, as well as equivalents thereof.

The purpose of the statements about the technical field is generally to enable the Patent and Trademark Office and the public to determine quickly, from a cursory inspection, the nature of this patent application. The description of the technical field is believed, at the time of the filing of this patent application, to adequately describe the technical field of this patent application. However, the description of the technical field may not be completely applicable to the claims as originally filed in this patent application, as amended during prosecution of this patent application, and as ultimately allowed in any patent issuing from this patent application. Therefore, any statements made relating to the technical field are not intended to limit the claims in any manner and should not be interpreted as limiting the claims in any manner.

The appended drawings in their entirety, including all dimensions, proportions and/or shapes in at least one embodiment of the invention, are accurate and are hereby included by reference into this specification.

The background information is believed, at the time of the filing of this patent application, to adequately provide background information for this patent application. However, the background information may not be completely applicable to the claims as originally filed in this patent application, as amended during prosecution of this patent application, and as ultimately allowed in any patent issuing from this patent application. Therefore, any statements made relating to the background information are not intended to limit the claims in any manner and should not be interpreted as limiting the claims in any manner.

All, or substantially all, of the components and methods of the various embodiments may be used with at least one embodiment or all of the embodiments, if more than one embodiment is described herein.

The purpose of the statements about the object or objects is generally to enable the Patent and Trademark Office and the public to determine quickly, from a cursory inspection, the nature of this patent application. The description of the object or objects is believed, at the time of the filing of this patent application, to adequately describe the object or objects of this patent application. However, the description of the object or objects may not be completely applicable to the claims as originally filed in this patent application, as amended during prosecution of this patent application, and as ultimately allowed in any patent issuing from this patent application. Therefore, any statements made relating to the object or objects are not intended to limit the claims in any manner and should not be interpreted as limiting the claims in any manner.

All of the patents, patent applications and publications recited herein, and in the Declaration attached hereto, are hereby incorporated by reference as if set forth in their entirety herein.

The summary is believed, at the time of the filing of this patent application, to adequately summarize this patent application. However, portions or all of the information contained in the summary may not be completely applicable to the claims as originally filed in this patent application, as amended during prosecution of this patent application, and as ultimately allowed in any patent issuing from this patent application. Therefore, any statements made relating to the summary are not intended to limit the claims in any manner and should not be interpreted as limiting the claims in any manner.

It will be understood that the examples of patents, published patent applications, and other documents which are included in this application and which are referred to in paragraphs which state “Some examples of . . . which may possibly be used in at least one possible embodiment of the present application . . . ” may possibly not be used or useable in any one or more embodiments of the application.

The sentence immediately above relates to patents, published patent applications and other documents either incorporated by reference or not incorporated by reference.

Some examples of methods and apparatuses for closing and/or ceiling beverage cans, which may possibly be utilized or adapted for use in at least one possible embodiment of the present application, may possibly be found in the following U.S. Pat. No. 6,915,553, having the title “Seaming apparatus and method for cans,” published on Jul. 12, 2005; No. 6,736,283, having the title “Can end, tooling for manufacture of the can end and seeming chuck adapted to affix a converted can end to a can body,” published on May 18, 2004; No. 6,561,004, having the title “Can lid closure and method of joining a can lid closure to a can body,” published on May 13, 2003; and No. 6,499,622, having the title “Can lid closure and method of joining a can lid closure to a can body,” published in Dec. 31, 2002.

Some examples of machines that place covers on beverage cans include the SAGA 150 Automatic Round Can Seamer, manufactured by JK Somme, located at Polígono Industrial Santecilla Pabellón n^{o} 4—Area 2 SANTECILLA, 09585 VALLE DE MENA —BURGOS, SPAIN; the American Can Canco 08 A Seamer, with the serial number 6824, manufactured by Hallmark Equipment, Inc., located at Hallmark Equipment, Inc., 11040 N. Monterey Rd., Morgan Hill, Calif. 95037-9362; and the CANCO 400 AUTOMATIC FOUR HEAD ROTARY CAN SEAMER, sold by Alard Equipment Company, located at 6483 Lake Avenue, PO Box 57, Williamson, N.Y. 14589-0057 U.S.A.

All of the patents, patent applications or patent publications, which were cited in the International Search Report dated Jul. 30, 2008, and/or cited elsewhere are hereby incorporated by reference as if set forth in their entirety herein as follows: JP 2005 170393, having the following English translation of the Japanese title “METHOD AND APPARATUS FOR STERILIZING CONTAINER,” published on Jun. 30, 2005; WO 99/30747, having the title “A CONTINUOUS PROCESS FOR HYPERACTIVATION OF FLUIDS FOR STERILIZATION,” published on Jun. 24, 1999; U.S. Pat. No. 4,742,667, having the title “Method of and apparatus for sterilizing packaging material, especially container-type packages,” published on May 10, 1988; US 2003/165400, having the title “Method of sterilization for container, apparatus using therefor and heat treatment for container,” published on Sep. 4, 2003; US 2003/230567, having the title “Vaporizer using electrical induction to produce heat,” published on Dec. 18, 2003; JP 11 278443, having the following English translation of the Japanese title “METHOD AND APPARATUS FOR HEATING CAN,” published on Oct. 12, 1999; and U.S. Pat. No. 3,961,150, having the title “Sterilization apparatus,” published on Jun. 1, 1976.

All of the patents, patent applications or patent publications, which were cited in the German Office Action dated Aug. 22, 2007, and/or cited elsewhere are hereby incorporated by reference as if set forth in their entirety herein as follows: EP 1,607,106, having the following English translation of the German title “Apparatus for sterilizing containers using H2O2,” published on Dec. 21, 2005; and DE 40 31 472, having the following English translation of the German title “Device for sterilizing, filling and closing of a filling opening,” published on Apr. 9, 1992.

An example of a device configured to inject hydrogen peroxide into a container for sterilization, which may possibly be utilized or adapted for use in at least one possible embodiment of the present application, may possibly be found in the following U.S. Pat. No. 7,010,900, having the title “Beverage bottling plant for filling bottles with a liquid beverage filling material, and a cleaning device for cleaning bottles in a beverage bottling plant,” published on Mar. 14, 2006.

The patents, patent applications, and patent publication listed above in the preceding five paragraphs are herein incorporated by reference as if set forth in their entirety. The purpose of incorporating U.S. patents, non-U.S. patents, publications, etc. is solely to provide additional information relating to technical features of one or more embodiments, which information may not be completely disclosed in the wording in the pages of this application. Words relating to the opinions and judgments of the author and not directly relating to the technical details of the description of the embodiments therein are not incorporated by reference. The words all, always, absolutely, consistently, preferably, guarantee, particularly, constantly, ensure, necessarily, immediately, endlessly, avoid, exactly, continually, expediently, need, must, only, perpetual, precise, perfect, require, requisite, simultaneous, total, unavoidable, and unnecessary, or words substantially equivalent to the above-mentioned words in this sentence, when not used to describe technical features of one or more embodiments, are not considered to be incorporated by reference herein.

The corresponding foreign and international patent publication applications, namely, Federal Republic of Germany Patent Application No. DE 10 2007 001 970.1, filed on Jan. 13, 2007, having inventor Wilhelm LOTHAR, and DE-OS 10 2007 001 970.1 and DE-PS 10 2007 001 970.1, and International Application No. PCT/EP2007/011235, filed on Dec. 20, 2007, having WIPO Publication No. WO 2008/083824 and inventor Wilhelm LOTHAR, are hereby incorporated by reference as if set forth in their entirety herein for the purpose of correcting and explaining any possible misinterpretations of the English translation thereof. In addition, the published equivalents of the above corresponding foreign and international patent publication applications, and other equivalents or corresponding applications, if any, in corresponding cases in the Federal Republic of Germany and elsewhere, and the references and documents cited in any of the documents cited herein, such as the patents, patent applications and publications, are hereby incorporated by reference as if set forth in their entirety herein.

The purpose of incorporating the corresponding foreign equivalent patent application(s), that is, PCT/EP2007/011235 and DE Patent Application 10 2007 001 970.1, is solely for the purpose of providing a basis of correction of any wording in the pages of the present application, which may have been mistranslated or misinterpreted by the translator. Words relating to opinions and judgments of the author and not directly relating to the technical details of the description of the embodiments therein are not to be incorporated by reference. The words all, always, absolutely, consistently, preferably, guarantee, particularly, constantly, ensure, necessarily, immediately, endlessly, avoid, exactly, continually, expediently, need, must, only, perpetual, precise, perfect, require, requisite, simultaneous, total, unavoidable, and unnecessary, or words substantially equivalent to the above-mentioned word in this sentence, when not used to describe technical features of one or more embodiments, are not generally considered to be incorporated by reference herein.

Statements made in the original foreign patent applications PCT/EP2007/011235 and DE 10 2007 001 970.1 from which this patent application claims priority which do not have to do with the correction of the translation in this patent application are not to be included in this patent application in the incorporation by reference.

Any statements about admissions of prior art in the original foreign patent applications PCT/EP2007/011235 and DE 10 2007 001 970.1 are not to be included in this patent application in the incorporation by reference, since the laws relating to prior art in non-U.S. Patent Offices and courts may be substantially different from the Patent Laws of the United States.

All of the references and documents, cited in any of the documents cited herein, are hereby incorporated by reference as if set forth in their entirety herein. All of the documents cited herein, referred to in the immediately preceding sentence, include all of the patents, patent applications and publications cited anywhere in the present application.

The description of the embodiment or embodiments is believed, at the time of the filing of this patent application, to adequately describe the embodiment or embodiments of this patent application. However, portions of the description of the embodiment or embodiments may not be completely applicable to the claims as originally filed in this patent application, as amended during prosecution of this patent application, and as ultimately allowed in any patent issuing from this patent application. Therefore, any statements made relating to the embodiment or embodiments are not intended to limit the claims in any manner and should not be interpreted as limiting the claims in any manner.

The details in the patents, patent applications and publications may be considered to be incorporable, at applicant's option, into the claims during prosecution as further limitations in the claims to patentably distinguish any amended claims from any applied prior art.

The purpose of the title of this patent application is generally to enable the Patent and Trademark Office and the public to determine quickly, from a cursory inspection, the nature of this patent application. The title is believed, at the time of the filing of this patent application, to adequately reflect the general nature of this patent application. However, the title may not be completely applicable to the technical field, the object or objects, the summary, the description of the embodiment or embodiments, and the claims as originally filed in this patent application, as amended during prosecution of this patent application, and as ultimately allowed in any patent issuing from this patent application. Therefore, the title is not intended to limit the claims in any manner and should not be interpreted as limiting the claims in any manner.

The abstract of the disclosure is submitted herewith as required by 37 C.F.R. §1.72(b). As stated in 37 C.F.R. §1.72(b):
A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims.
   Therefore, any statements made relating to the abstract are not intended to limit the claims in any manner and should not be interpreted as limiting the claims in any manner.

The embodiments of the invention described herein above in the context of the preferred embodiments are not to be taken as limiting the embodiments of the invention to all of the provided details thereof, since modifications and variations thereof may be made without departing from the spirit and scope of the embodiments of the invention.

### AT LEAST PARTIAL NOMENCLATURE

1 Conveyor device
2 Sterilization carousel
3 Sterilization heads
4 Transport device
5 Reaction line
6 Induction coil arrangements
7 Air feed
8 Exhaust device
9 Conveyor device
10 Filler
11 Transport device
21 Conveyor device
22 Sterilization carousel
23 Vaporizer heads
24 Reaction carousel
25 Feed nozzles
26 Transport star wheel
27 Filler

What is claimed is:

1. A method of treating beverage cans in a beverage can filling plant, said method comprising the steps of:
moving a beverage can, with a first conveyor device, into a rotary beverage can treating machine, which rotary beverage can treating machine is configured to inject a treating agent into beverage cans;
injecting, into said beverage can, an amount of treating agent sufficient to condense on at least substantially all the inner surfaces of a beverage can, with one of a plurality of treating agent injection heads disposed on said rotary beverage can treating machine, which amount of treating agent is further sufficient to permit sterilization of beverage cans;
moving said beverage can, in said rotary beverage can treating machine, for a sufficient period of time to permit said sufficient amount of injected treated agent to at least initiate sufficient condensation on said at least substantially all the inner surfaces of said beverage can;
permitting said sufficient amount of injected treating agent to condense on said at least substantially all the inner surfaces of said beverage can;
moving said beverage can, with said sufficient amount of injected treating agent condensed on said at least substantially all the inner surfaces, out of said rotary beverage can treating machine, with a second conveyor device;
moving said beverage can, with said sufficient amount of injected treating agent condensed on said at least substantially all the inner surfaces, into a reaction tunnel, with said second conveyor device;
moving said beverage can, with said sufficient amount of injected treating agent condensed on said at least substantially all the inner surfaces, through said reaction tunnel in a direction of transport;
heating said beverage can, with said sufficient amount of injected treating agent condensed on said at least substantially all the inner surfaces, with an induction coil arrangement;
heating said sufficient amount of injected treating agent condensed on said at least substantially all the inner surfaces of said beverage can;
activating said sufficient amount of injected treating agent condensed on said at least substantially all the inner surfaces of said beverage can;
treating said beverage can while moving said beverage can through said reaction tunnel;
moving said beverage can, through said reaction tunnel, for a sufficient period of time to permit said sufficient amount of injected treated agent to sufficiently treat said beverage can;
introducing sterile air into said reaction tunnel with an air feed, which air feed is connected to said reaction tunnel on the end farther from said second conveyor device;
removing sterile air out of said reaction tunnel with an exhaust device, which exhaust device is connected to said reaction tunnel on the end closer to said second conveyor device;
flowing sterile air through said reaction tunnel against said direction of transport of said beverage cans and removing any gaseous and any other decomposition products formed by said steps of activating and treating;
moving said treated beverage can out of said reaction tunnel, with a third conveyor device;
moving said treated beverage can, with said third conveyor device, into a rotary beverage can filling machine;
filling said treated beverage can with said rotary beverage can filling machine;
moving said filled, treated beverage can out of said rotary beverage can filling machine, with a fourth conveyor device;
moving said filled, treated beverage can into a rotary beverage can closing machine with said fourth conveyor device;
applying, to a beverage can cover, an amount of treating agent sufficient to condense on at least the inside of said beverage can cover;
permitting said sufficient amount of treating agent to condense on said at least the inside of said beverage can cover;
heating said sufficient amount of treating agent on said at least the inside of said beverage can cover;
activating said sufficient amount of treating agent on said at least the inside of said beverage can cover;
treating said beverage can cover;
moving said treated beverage can cover into said rotary beverage can closing machine; and
closing said filled, treated beverage can with said treated beverage can cover.

2. The method of treating beverage cans in a beverage can filling plant according to claim 1, wherein said method further comprises one of (A) and (B):
(A) said step of injecting further comprises injecting said treating agent in liquid form; and
(B) said step of injecting further comprises injecting said treating agent in vapor form with sterile air.

3. The method of treating beverage cans in a beverage can filling plant according to claim 2, wherein:
said treating agent further comprises hydrogen peroxide; and
said method further comprises:
sterilizing said beverage can; and
sterilizing said beverage can cover.

4. Means for performing the method of treating beverage cans in a beverage can filling plant according to claim 1, said means comprising:
means for moving a beverage can, with a first conveyor device, into a rotary beverage can treating machine, which rotary beverage can treating machine is configured to inject a treating agent into beverage cans;
means for injecting, into a beverage can, an amount of treating agent sufficient to condense on at least substantially all the inner surfaces of a beverage can, with one of a plurality of treating agent injection heads disposed on said rotary beverage can treating machine, which amount of treating agent is further sufficient to permit sterilization of beverage cans;
means for moving a beverage can, in said rotary beverage can treating machine, for a sufficient period of time to permit a sufficient amount of injected treated agent to at least initiate sufficient condensation on at least substantially all the inner surfaces of the beverage can;
means for permitting a sufficient amount of injected treating agent to condense on at least substantially all the inner surfaces of a beverage can;
means for moving a beverage can, with a sufficient amount of injected treating agent condensed on at least substantially all the inner surfaces, out of said rotary beverage can treating machine, with a second conveyor device;
means for moving a beverage can, with a sufficient amount of injected treating agent condensed on at least substantially all the inner surfaces, into a reaction tunnel, with said second conveyor device;
means for moving a beverage can, with a sufficient amount of injected treating agent condensed on at least substantially all the inner surfaces, through said reaction tunnel in a direction of transport;
means for heating a beverage can, with a sufficient amount of injected treating agent condensed on at least substantially all the inner surfaces, with an induction coil arrangement;
means for heating a sufficient amount of injected treating agent condensed on at least substantially all the inner surfaces of a beverage can;
means for activating a sufficient amount of injected treating agent condensed on at least substantially all the inner surfaces of a beverage can;
means for treating a beverage can while moving the beverage can through said reaction tunnel;
means for moving a beverage can, through said reaction tunnel, for a sufficient period of time to permit a sufficient amount of injected treated agent to sufficiently treat the beverage can;
means for introducing sterile air into said reaction tunnel with an air feed, which air feed is connected to said reaction tunnel on the end farther from said second conveyor device;
means for removing sterile air out of said reaction tunnel with an exhaust device, which exhaust device is connected to said reaction tunnel on the end closer to said second conveyor device;
means for flowing sterile air through said reaction tunnel against a direction of transport of beverage cans and removing any gaseous and any other decomposition products formed by activating and treating;
means for moving a treated beverage can out of said reaction tunnel, with a third conveyor device;
means for moving a treated beverage can, with said third conveyor device, into a rotary beverage can filling machine;
means for filling a treated beverage can with said rotary beverage can filling machine;
means for moving a filled, treated beverage can out of said rotary beverage can filling machine, with a fourth conveyor device;
means for moving a filled, treated beverage can into a rotary beverage can closing machine with said fourth conveyor device;
means for applying, to a beverage can cover, an amount of treating agent sufficient to condense on at least the inside of the beverage can cover;
means for permitting a sufficient amount of treating agent to condense on at least the inside of a beverage can cover;
means for heating a sufficient amount of treating agent on at least the inside of a beverage can cover;
means for activating a sufficient amount of treating agent on at least the inside of a beverage can cover;
means for treating a beverage can cover;
means for moving a treated beverage can cover into said rotary beverage can closing machine; and
means for closing a filled, treated beverage can with a treated beverage can cover.

5. A beverage can treatment arrangement for performing the method of treating beverage cans in a beverage can filling plant according to claim 1, said beverage can treatment arrangement comprising:
a first moving arrangement being configured to move a beverage can, with a first conveyor device, into a rotary beverage can treating machine, which rotary beverage can treating machine is configured to inject a treating agent into beverage cans;
an injecting arrangement being configured to inject, into a beverage can, an amount of treating agent sufficient to condense on at least substantially all the inner surfaces of a beverage can, with one of a plurality of treating agent injection heads disposed on said rotary beverage can treating machine, which amount of treating agent is further sufficient to permit sterilization of beverage cans;
a second moving arrangement being configured to move a beverage can, in said rotary beverage can treating machine, for a sufficient period of time to permit a sufficient amount of injected treated agent to at least initiate sufficient condensation on at least substantially all the inner surfaces of the beverage can;
a first permitting arrangement being configured to permit a sufficient amount of injected treating agent to condense on at least substantially all the inner surfaces of a beverage can;
a third moving arrangement being configured to move a beverage can, with a sufficient amount of injected treating agent condensed on at least substantially all the inner surfaces, out of said rotary beverage can treating machine, with a second conveyor device;
said third moving arrangement being configured to move a beverage can, with a sufficient amount of injected treating agent condensed on at least substantially all the inner surfaces, into a reaction tunnel, with said second conveyor device;
a fourth moving arrangement being configured to move a beverage can, with a sufficient amount of injected treating agent condensed on at least substantially all the inner surfaces, through said reaction tunnel in a direction of transport;
a first heating arrangement being configured to heat a beverage can, with a sufficient amount of injected treating agent condensed on at least substantially all the inner surfaces, with an induction coil arrangement;
said first heating arrangement being further configured to heat a sufficient amount of injected treating agent condensed on at least substantially all the inner surfaces of a beverage can;
a first activating arrangement being configured to activate a sufficient amount of injected treating agent condensed on at least substantially all the inner surfaces of a beverage can;
a first treating arrangement being configured to treat a beverage can while moving the beverage can through said reaction tunnel;
a fifth moving arrangement being configured to move a beverage can, through said reaction tunnel, for a sufficient period of time to permit a sufficient amount of injected treated agent to sufficiently treat the beverage can;
an introducing arrangement being configured to introduce sterile air into said reaction tunnel with an air feed, which air feed is connected to said reaction tunnel on the end farther from said second conveyor device;
a removing arrangement being configured to remove sterile air out of said reaction tunnel with an exhaust device, which exhaust device is connected to said reaction tunnel on the end closer to said second conveyor device;
a flowing arrangement being configured to flow sterile air through said reaction tunnel against a direction of transport of beverage cans and removing any gaseous and any other decomposition products formed by activating and treating;
a sixth moving arrangement being configured to move a treated beverage can out of said reaction tunnel, with a third conveyor device;
said sixth moving arrangement being configured to move a treated beverage can, with said third conveyor device, into a rotary beverage can filling machine;
a filling arrangement being configured to fill a treated beverage can with said rotary beverage can filling machine;
a seventh moving arrangement being configured to move a filled, treated beverage can out of said rotary beverage can filling machine, with a fourth conveyor device;
said seventh moving arrangement being configured to move a filled, treated beverage can into a rotary beverage can closing machine with said fourth conveyor device;
an applying arrangement being configured to apply, to a beverage can cover, an amount of treating agent sufficient to condense on at least the inside of the beverage can cover;
a second permitting arrangement being configured to permit a sufficient amount of treating agent to condense on at least the inside of a beverage can cover;
a second heating arrangement being configured to heat a sufficient amount of treating agent on at least the inside of a beverage can cover;
a second activating arrangement being configured to activate a sufficient amount of treating agent on at least the inside of a beverage can cover;
a second treating arrangement being configured to treat a beverage can cover;
an eighth moving arrangement being configured to move a treated beverage can cover into said rotary beverage can closing machine; and
a closing arrangement being configured to close a filled, treated beverage can with a treated beverage can cover.

6. A method of sterilizing beverage cans in a beverage can filling plant, said method comprising the steps of:
moving a beverage can, with a first conveyor device, into a rotary beverage can sterilizing machine, which rotary beverage can sterilizing machine is configured to inject hydrogen peroxide into beverage cans;
injecting, into said beverage can, an amount of hydrogen peroxide sufficient to condense on at least substantially all the inner surfaces of a beverage can, with one of a plurality of sterilization heads disposed on rotary said beverage can sterilizing machine, which amount of treating agent is further sufficient to permit sterilization of beverage cans;
moving said beverage can, in said rotary beverage can treating machine, for a sufficient period of time to permit said sufficient amount of injected treated agent to at least initiate sufficient condensation on said at least substantially all the inner surfaces of said beverage can;
permitting said sufficient amount of injected hydrogen peroxide to condense on said at least substantially all the inner surfaces of said beverage can;
moving said beverage can, with said sufficient amount of injected hydrogen peroxide condensed on said at least substantially all the inner surfaces, out of said rotary beverage can sterilizing machine, with a second conveyor device;
moving said beverage can, with said sufficient amount of injected hydrogen peroxide condensed on said at least substantially all the inner surfaces, into a rotary reaction machine, with said second conveyor device;
blowing heated sterile air into said beverage can with one of a plurality of feed nozzles disposed on said rotary reaction machine;
heating, in said rotary reaction machine, said sufficient amount of injected hydrogen peroxide condensed on said at least substantially all the inner surfaces of said beverage can with the heated sterile air;
activating said sufficient amount of injected hydrogen peroxide condensed on said at least substantially all the inner surfaces of said beverage can;
sterilizing said beverage can while moving said beverage can through said rotary reaction machine;
moving said beverage can, through said reaction machine, for a sufficient period of time to permit said sufficient amount of injected treated agent to sufficiently treat said beverage can;
moving said sterilized beverage can out of said rotary reaction machine, with a third conveyor device;
moving said sterilized beverage can, with said third conveyor device, into a rotary beverage can filling machine;
filling said sterilized beverage can with said rotary beverage can filling machine; and
moving said filled, sterilized beverage can out of said rotary beverage can filling machine, with a fourth conveyor device;
moving said filled, treated beverage can into a rotary beverage can closing machine with said fourth conveyor device;
applying, to a beverage can cover, an amount of treating agent sufficient to condense on at least the inside of said beverage can cover;
permitting said sufficient amount of treating agent to condense on said at least the inside of said beverage can cover;
heating said sufficient amount of treating agent on said at least the inside of said beverage can cover;
activating said sufficient amount of treating agent on said at least the inside of said beverage can cover;
treating said beverage can cover;
moving said treated beverage can cover into said rotary beverage can closing machine; and
closing said filled, treated beverage can with said treated metal can cover.

7. The method of sterilizing beverage cans in a beverage can filling plant according to claim 6, wherein:
said method further comprises one of (A) and (B):
(A) said step of injecting further comprises injecting said hydrogen peroxide in liquid form; and
(B) said step of injecting further comprises injecting said hydrogen peroxide in vapor form with sterile air.

8. The method of sterilizing beverage cans in a beverage can filling plant according to claim 7, wherein said method further comprises flowing sterile air through said reaction machine against the direction of transport of said beverage can and removing any gaseous and any other decomposition products formed by said steps of activating and sterilizing.

9. Means for performing the method of sterilizing beverage cans in a beverage can filling plant according to claim 6, said means comprising:
means for moving a beverage can, with a first conveyor device, into a rotary beverage can sterilizing machine, which rotary beverage can sterilizing machine is configured to inject hydrogen peroxide into beverage cans;
means for injecting, into a beverage can, an amount of hydrogen peroxide sufficient to condense on at least substantially all the inner surfaces of a beverage can, with one of a plurality of sterilization heads disposed on rotary said beverage can sterilizing machine, which amount of treating agent is further sufficient to permit sterilization of beverage cans;
means for moving a beverage can, in said rotary beverage can treating machine, for a sufficient period of time to permit a sufficient amount of injected treated agent to at least initiate sufficient condensation on at least substantially all the inner surfaces of the beverage can;
means for permitting a sufficient amount of injected hydrogen peroxide to condense on at least substantially all the inner surfaces of a beverage can;
means for moving a beverage can, with a sufficient amount of injected hydrogen peroxide condensed on at least substantially all the inner surfaces, out of said rotary beverage can sterilizing machine, with a second conveyor device;
means for moving a beverage can, with a sufficient amount of injected hydrogen peroxide condensed on at least substantially all the inner surfaces, into a rotary reaction machine, with said second conveyor device;
means for blowing heated sterile air into a beverage can with one of a plurality of feed nozzles disposed on said rotary reaction machine;
means for heating, in said rotary reaction machine, a sufficient amount of injected hydrogen peroxide condensed on at least substantially all the inner surfaces of a beverage can with the heated sterile air;
means for activating a sufficient amount of injected hydrogen peroxide condensed on at least substantially all the inner surfaces of a beverage can;
means for sterilizing a beverage can while moving the beverage can through said rotary reaction machine;
means for moving a beverage can, through said reaction machine, for a sufficient period of time to permit a sufficient amount of injected treated agent to sufficiently treat the beverage can;
means for moving a sterilized beverage can out of said rotary reaction machine, with a third conveyor device;
means for moving a sterilized beverage can, with said third conveyor device, into a rotary beverage can filling machine;
means for filling a sterilized beverage can with said rotary beverage can filling machine;
means for moving a filled, sterilized beverage can out of said rotary beverage can filling machine, with a fourth conveyor device;
means for moving a filled, treated beverage can into a rotary beverage can closing machine with said fourth conveyor device;
means for applying, to a beverage can cover, an amount of treating agent sufficient to condense on at least the inside of the beverage can cover;
means for permitting a sufficient amount of treating agent to condense on at least the inside of a beverage can cover;
means for heating a sufficient amount of treating agent on at least the inside of a beverage can cover;
means for activating a sufficient amount of treating agent on at least the inside of a beverage can cover;
means for treating a beverage can cover;
means for moving a treated beverage can cover into said rotary beverage can closing machine; and
means for closing a filled, treated beverage can with a treated metal can cover.

10. A beverage can sterilizing arrangement for performing the method of sterilizing beverage cans in a beverage can filling plant according to claim 6, said beverage can sterilizing arrangement comprising:
a first moving arrangement being configured to move a beverage can, with a first conveyor device, into a rotary beverage can sterilizing machine, which rotary beverage can sterilizing machine is configured to inject hydrogen peroxide into beverage cans;
an injecting means being configured to inject, into a beverage can, an amount of hydrogen peroxide sufficient to condense on at least substantially all the inner surfaces of a beverage can, with one of a plurality of sterilization heads disposed on rotary said beverage can sterilizing machine, which amount of treating agent is further sufficient to permit sterilization of beverage cans;
a second moving arrangement being configured to move a beverage can, in said rotary beverage can treating machine, for a sufficient period of time to permit a sufficient amount of injected treated agent to at least initiate sufficient condensation on at least substantially all the inner surfaces of the beverage can;
a first permitting arrangement being configured to permit a sufficient amount of injected hydrogen peroxide to condense on at least substantially all the inner surfaces of a beverage can;
a third moving arrangement being configured to move a beverage can, with a sufficient amount of injected hydrogen peroxide condensed on at least substantially all the inner surfaces, out of said rotary beverage can sterilizing machine, with a second conveyor device;
said third moving arrangement being configured to move a beverage can, with a sufficient amount of injected hydrogen peroxide condensed on at least substantially all the inner surfaces, into a rotary reaction machine, with said second conveyor device;
a blowing arrangement being configured to blow heated sterile air into a beverage can with one of a plurality of feed nozzles disposed on said rotary reaction machine;
a first heating arrangement being configured to heat, in said rotary reaction machine, a sufficient amount of injected hydrogen peroxide condensed on at least substantially all the inner surfaces of a beverage can with the heated sterile air;
a first activating arrangement being configured to activate a sufficient amount of injected hydrogen peroxide condensed on at least substantially all the inner surfaces of a beverage can;
a first sterilizing arrangement being configured to sterilize a beverage can while moving the beverage can through said rotary reaction machine;
a fourth moving arrangement being configured to move a beverage can, through said reaction machine, for a sufficient period of time to permit a sufficient amount of injected treated agent to sufficiently treat the beverage can;
a fifth moving arrangement being configured to move a sterilized beverage can out of said rotary reaction machine, with a third conveyor device;
said fifth moving arrangement being configured to move a sterilized beverage can, with said third conveyor device, into a rotary beverage can filling machine;
a filling arrangement being configured to fill a sterilized beverage can with said rotary beverage can filling machine;
a sixth moving arrangement being configured to move a filled, sterilized beverage can out of said rotary beverage can filling machine, with a fourth conveyor device;
said sixth moving arrangement being configured to move a filled, treated beverage can into a rotary beverage can closing machine with said fourth conveyor device;
an applying arrangement being configured to apply, to a beverage can cover, an amount of treating agent sufficient to condense on at least the inside of the beverage can cover;
a second permitting arrangement being configured to permit a sufficient amount of treating agent to condense on at least the inside of a beverage can cover;
a second heating arrangement being configured to heat a sufficient amount of treating agent on at least the inside of a beverage can cover;
a second activating arrangement being configured to activate a sufficient amount of treating agent on at least the inside of a beverage can cover;
a second treating arrangement being configured to treat a beverage can cover;
a seventh moving arrangement being configured to move a treated beverage can cover into said rotary beverage can closing machine; and
a closing arrangement being configured to close a filled, treated beverage can with a treated metal can cover.

11. A method of treating metal cans in a metal can filling plant, said method comprising the steps of:
moving a metal can, with a first conveyor device, into a metal can treating machine, which metal can treating machine is configured to inject a treating agent into metal cans;
injecting, into said metal can, an amount of treating agent sufficient to condense on at least substantially all the inner surfaces of a metal can, with one of a plurality of treating agent injection heads disposed on said metal can treating machine, which amount of treating agent is further sufficient to permit sterilization of metal cans;
moving said metal can, in said rotary metal can treating machine, for a sufficient period of time to permit said sufficient amount of injected treated agent to sufficiently condense on said at least substantially all the inner surfaces of said metal can;
permitting said sufficient amount of injected treating agent to condense on said at least substantially all the inner surfaces of said metal can;
moving said metal can, with said sufficient amount of injected treating agent condensed on said at least substantially all the inner surfaces, out of said metal can treating machine, with a second conveyor device;
moving said metal can, with said sufficient amount of injected treating agent condensed on said at least substantially all the inner surfaces, into a reaction arrangement, with said second conveyor device;
activating said sufficient amount of injected treating agent condensed on said at least substantially all the inner surfaces of said metal can;
treating said metal can while moving said metal can through said reaction arrangement and sterilizing said metal can;
moving said metal can, through said reaction arrangement, for a sufficient period of time to permit said sufficient amount of injected treated agent to sufficiently treat said metal can;
moving said treated metal can out of said reaction arrangement, with a third conveyor device;
moving said treated metal can, with said third conveyor device, into a metal can filling machine;
filling said treated metal can with said metal can filling machine; and
moving said filled, treated metal can out of said metal can filling machine, with a fourth conveyor device.

12. The method of treating metal cans in a metal can filling plant according to claim 11, wherein said method further comprises the steps of:
moving said filled, treated metal can into a metal can closing machine with said fourth conveyor device;
applying, to a metal can cover, an amount of treating agent sufficient to condense on at least the inside of said metal can cover;
permitting said sufficient amount of treating agent to condense on said at least the inside of said metal can cover;
activating said sufficient amount of treating agent on said at least the inside of said metal can cover;
treating said metal can cover;
moving said treated metal can cover into said metal can closing machine; and
closing said filled, treated metal can with said treated metal can cover.

13. The method of treating metal cans in a metal can filling plant according to claim 12, wherein said method further comprises one of (A) and (B):
(A) said step of injecting further comprises injecting said treating agent in liquid form; and
(B) said step of injecting further comprises injecting said treating agent in vapor form with sterile air.

14. The method of treating metal cans in a metal can filling plant according to claim 13, wherein said treating agent further comprises hydrogen peroxide.

15. The method of treating metal cans in a metal can filling plant according to claim 14, wherein said step of activating further comprises heating said sufficient amount of injected treating agent condensed on said at least substantially all the inner surfaces of said metal can and sterilizing said metal can.

16. The method of treating metal cans in a metal can filling plant according to claim 15, wherein said step of activating said sufficient amount of treating agent on said at least the inside of said metal can cover further comprises heating said sufficient amount of treating agent on said at least the inside of said metal can cover.

17. The method of treating metal cans in a metal can filling plant according to claim 16, wherein one of (C) and (D):
(C) said step of heating said sufficient amount of injected treating agent condensed on said at least substantially all the inner surfaces of said metal can further comprises blowing heated sterile air into said metal can with one of a plurality of feed nozzles disposed on said reaction arrangement; and
(D) said step of heating said sufficient amount of injected treating agent condensed on said at least substantially all the inner surfaces of said metal can further comprises heating said metal can, with said sufficient amount of injected treating agent condensed on said at least substantially all the inner surfaces, with an induction coil arrangement, and heating said sufficient amount of injected treating agent condensed on said at least substantially all the inner surfaces of said metal can.

18. The method of treating metal cans in a metal can filling plant according to claim 17, wherein said method further comprises flowing sterile air through said reaction arrangement against the direction of transport of said metal can and removing any gaseous and any other decomposition products formed by said steps of activating and treating.

19. Means for performing the method of treating metal cans in a metal can filling plant according to claim 11, said means comprising:
means for moving a metal can, with a first conveyor device, into a metal can treating machine, which metal can treating machine is configured to inject a treating agent into metal cans;
means for injecting, into a metal can, an amount of treating agent sufficient to condense on at least substantially all the inner surfaces of a metal can, with one of a plurality of treating agent injection heads disposed on said metal can treating machine, which amount of treating agent is further sufficient to permit sterilization of metal cans;
means for moving a metal can, in said rotary metal can treating machine, for a sufficient period of time to permit the sufficient amount of injected treated agent to sufficiently condense on at least substantially all the inner surfaces of the metal can;
means for permitting a sufficient amount of injected treating agent to condense on at least substantially all the inner surfaces of a metal can;
means for moving a metal can, with a sufficient amount of injected treating agent condensed on at least substantially all the inner surfaces, out of said metal can treating machine, with a second conveyor device;
means for moving a metal can, with a sufficient amount of injected treating agent condensed on at least substantially all the inner surfaces, into a reaction arrangement, with said second conveyor device;
means for activating a sufficient amount of injected treating agent condensed on at least substantially all the inner surfaces of a metal can;
means for treating a metal can while moving the metal can through said reaction arrangement and sterilizing the metal can;
means for moving a metal can, through said reaction arrangement, for a sufficient period of time to permit a sufficient amount of injected treated agent to sufficiently treat the metal can;
means for moving a treated metal can out of said reaction arrangement, with a third conveyor device;
means for moving a treated metal can, with said third conveyor device, into a metal can filling machine;
means for filling a treated metal can with said metal can filling machine; and
means for moving a filled, treated metal can out of said metal can filling machine, with a fourth conveyor device.

20. A metal can treatment arrangement for performing the method of treating metal cans in a metal can filling plant according to Claim 11, said metal can treatment arrangement comprising:
a first moving arrangement being configured to move a metal can, with a first conveyor device, into a metal can treating machine, which metal can treating machine is configured to inject a treating agent into metal cans;
an injecting arrangement being configured to inject, into a metal can, an amount of treating agent sufficient to condense on at least substantially all the inner surfaces of a metal can, with one of a plurality of treating agent injection heads disposed on said metal can treating machine, which amount of treating agent is further sufficient to permit sterilization of metal cans;
a second moving arrangement being configured to move a metal can, in said rotary metal can treating machine, for a sufficient period of time to permit the sufficient amount of injected treated agent to sufficiently condense on at least substantially all the inner surfaces of the metal can;
a permitting arrangement being configured to permit a sufficient amount of injected treating agent to condense on at least substantially all the inner surfaces of a metal can;
a third moving arrangement being configured to move a metal can, with a sufficient amount of injected treating agent condensed on at least substantially all the inner surfaces, out of said metal can treating machine, with a second conveyor device;
said third moving arrangement being configured to move a metal can, with a sufficient amount of injected treating agent condensed on at least substantially all the inner surfaces, into a reaction arrangement, with said second conveyor device;
an activating arrangement being configured to activate a sufficient amount of injected treating agent condensed on at least substantially all the inner surfaces of a metal can;
a treating arrangement being configured to treat a metal can while moving the metal can through said reaction arrangement and sterilize the metal can;
a fourth moving arrangement being configure to move a metal can, through said reaction arrangement, for a sufficient period of time to permit a sufficient amount of injected treated agent to sufficiently treat the metal can;
a fifth moving arrangement being configured to move a treated metal can out of said reaction arrangement, with a third conveyor device;
said fifth moving arrangement being configured to move a treated metal can, with said third conveyor device, into a metal can filling machine;
a filling arrangement being configured to fill a treated metal can with said metal can filling machine; and
a sixth moving arrangement being configured to move a filled, treated metal can out of said metal can filling machine, with a fourth conveyor device.
